(19) United States
(12) Patent Application Publication
(10) Pub. No.: US 2008/0279328 A1
(43) Pub. Date: Nov. 13, 2008

(54) Systems and Methods Using X-Ray Tube Spectra For Computed Tomography Applications

(75) Inventors: Guenter Zeitler, Aachen (DE); Christoph Herrmann, Aachen (DE); Klaus Engel, Aachen (DE); Christian Baeumer, Aachen (DE); Ewald Roessl, Hamburg (DE); Roland Proksa, Hamburg (DE)

Correspondence Address: PHILIPS INTELLECTUAL PROPERTY & STANDARDS, 595 MINER ROAD, CLEVELAND, OH, 44143, US

(73) Assignee: Koninklijke Philips Electronics N.V., Eindoven (NL)

(21) Appl. No.: 12/093,186

(22) PCT Filed: Nov. 14, 2006
(86) PCT No.: PCT/IB06/54246

§ 371 Date: May 9, 2008

Related U.S. Application Data
(60) Provisional application No. 60/738,239, filed on Nov. 18, 2005.

(51) Int. Cl. A61B 6/00

(52) U.S. Cl. 378/4

(57) ABSTRACT

Computed tomography (CT) systems are provided that utilize x-ray tube spectra in connection with the generation and/or interpretation of CT data. The disclosed systems and methods use x-ray tube spectra associated with CT systems to enhance contrast and/or image quality, e.g., by making use of energy selective detection techniques. The x-ray spectra may be determined in a variety of ways, e.g., incorporation of a spectral x-ray tube model into the CT system, using the output of Monte-Carlo simulations, and/or processing measured experimental spectral tube data for the CT system. The x-ray tube spectra is generally generated by and/or stored in a computer system associated with the CT system and may be used in support of an energy selective detective method and/or generation of spectral CT images.

The present disclosure is directed to computed tomography (CT) systems that utilize energy properties and x-ray tube spectra in connection with the generation and/or interpretation of CT data. More particularly, the present disclosure is directed to systems and methods for using energy properties and/or x-ray tube spectra associated with CT systems to enhance contrast and/or image quality. The disclosed energy properties and x-ray spectra may be determined in a variety of ways, e.g., through incorporation of a spectral x-ray tube model into the CT system, using the output of Monte-Carlo simulations, and/or processing measured experimental spectral tube data for the CT system.

Computed tomography (CT) systems use x-rays to produce detailed images/pictures of internal anatomical structures. Generally, a CT system directs a series of x-ray pulses through the body. Each x-ray pulse generally lasts only a fraction of a second and represents a projection. After reconstruction, a set of projections is called a “slice” of the organ or area being studied. The slices or pictures are recorded on a computer and can be saved for further study or printed out as photographs. Dense tissue, such as bone, appear white on a typical CT image while less dense tissue, e.g., brain tissue or muscle, generally appear in shades of gray. Air-filled spaces, e.g., in the bowel or lung, appear black. CT scans can be used to obtain information about a wide variety of anatomical structures, e.g., the liver, pancreas, intestines, kidneys, adrenal glands, lungs, and heart, blood vessels, the abdominal cavity, bones, and the spinal cord.

CT imaging typically employs an x-ray source that generates a fan-beam or cone-beam of x-rays that traverse an examination region. A subject positioned in the examination region interacts with and absorbs a portion of the traversing x-rays. Standard x-ray sources include a single cathode that emits an electron beam, which is accelerated and focused onto a single focus on an anode. Upon collision with the anode, a small fraction of the incident electron energy is converted into x-rays. A large percentage of the incident energy is translated to heat and deposited in the anode. To prevent anode damage due to the incident heat, the anode typically takes the form of a rotating disk, thereby defining a relative velocity between the incident electron beam and the anode surface (referred to as the “track velocity”). Generally, the higher the track velocity associated with a CT system, the higher the power density that can be obtained from the CT system. Although the track velocity can be increased by increasing the radius of the anode disk and/or by increasing it rotation speed/frequency, the technical limits for such approaches to increasing power density have been approached, if not reached.

A CT data measurement system (DMS) generally includes a two-dimensional detector array arranged opposite the x-ray source to detect and measure intensities of the transmitted x-rays. Typically, the x-ray source and the DMS are mounted at opposite sides of a rotating gantry. As the gantry is rotated, an angular range of projection views of the subject are obtained.

The two-dimensional detector array of the DMS typically includes a scintillator crystal or array of scintillators which produce bursts of light, called scintillation events, responsive to impingement of x-rays onto the scintillator. A two-dimensional array of photodetectors, such as photodiodes or photomultiplier tubes, are arranged to view the scintillator and produce analog electrical signals in response to the scintillation events. The analog electrical signals are routed via electrical cabling to an analog-to-digital converter which digitizes the analog signals. The digitized signals are multiplexed into a reduced number of transmission channels, and the transmission channels communicate the multiplexed digitized signals.

Various techniques for energy selective CT (Spectral CT) imaging operation are known. For example, such CT systems may employ the conventionally used integration mode of operation. Advanced integrating modes perform, e.g., tube switching or, e.g., utilize detectors having multiple layers with different energy selectivity. Counting modes of operation—which are not state-of-the-art in CT yet—are also known, e.g., a combination of counting and integrating modes, energy weighting and energy binning (windowing) techniques. However, in the design and operation of conventional CT systems, the potential implications of the energy properties, e.g. through x-ray photon cross section data (esp. for Compton and Photoeffect), and, particularly, the x-ray tube spectra of specific CT unit(s) have not been taken into consideration—especially not the beneficial knowledge of the incident (filtered) x-ray tube spectrum to which the patient is exposed.

U.S. Patent Publication US 2004/0066908 to Hanke et al. describes a system for replacing measured data with simulated data, e.g., when high absorption density yields erroneous and/or incomplete projection data. The Hanke '908 publication accesses a model of the device-under-study from computer memory, e.g., CAD data. The stored information indicates local material densities, target geometry and other material properties. The Hanke '908 system employs a simulator that utilizes the stored information regarding the device-under-study and parameters concerning the CT system (which are stored in a different computer memory), e.g., transmitted primary x-ray spectrum of the x-ray emitter and detector characteristics, to generate simulated CT data. The simulated data is used to determine the measuring parameters for the device-under-study, e.g., measuring positions and transmission directions, and to supplement the measured data, e.g., if projection data is missing from the measured data or is inaccurate/highly noisy. For purposes of medical applications, the Hanke '908 publication discloses utility for the reduction of metal artifacts (e.g., due to metal implants).

U.S. Pat. No. 6,222,907 to Gordon, III, et al. discloses an approach to optimizing image quality in x-ray systems through generation of a x-ray technique trajectory. The Gordon '907 patent involves determining optimized x-ray techniques for a fixed spectral filter and focal spot to define a basic trajectory, optimizing the spectral filter and focal spot versus patient size, and combining the determined optimized techniques for a fixed spectral filter and focal spot with the optimized spectral filter and focal spot versus patient size, to create a functional trajectory.

Despite efforts to date, a need remains for CT systems that effectively address the implications of the energy properties of CT units. More particularly, a need remains for CT systems that effectively address the implications of the x-ray tube spectra of CT units. Additionally, a need remains for CT systems that access and/or use the energy properties and/or x-ray tube spectra of CT units to improve CT performance, e.g., the contrast and image quality associated therewith.

According to the present disclosure, computed tomography (CT) systems are provided that utilize energy properties and/or x-ray tube spectra of CT units to enhance CT performance, e.g., in generating and/or interpreting CT data. Indeed, in exemplary embodiments of the present disclosure, CT systems and methods are disclosed for using energy properties and/or x-ray tube spectra associated with CT systems to enhance contrast and/or image quality, e.g., by making use of advantageous energy selective detection techniques. An exemplary energy selective detection technique is described by Alvarez and Macovski, “Energy-Selective Reconstructions in X-ray Computerized Tomography,” Phys. Med. Biol., 1976 (the “Alvarez-Macovski approach”). The entire contents of the foregoing article by Alvarez and Macovski are incorporated herein by reference. The disclosed CT systems may be adapted to determine applicable energy properties and/or x-ray spectra in a variety of ways, e.g., through incorporation of a spectral x-ray tube model into the CT system, using the output of Monte-Carlo simulations, and/or processing measured experimental spectral tube data for the CT system.

According to exemplary embodiments of the present disclosure, a CT system is provided that includes an x-ray tube for directing an x-ray beam toward a structure, e.g., a patient, and a detector array positioned opposite the x-ray tube. The x-ray tube and detector array are generally mounted on a gantry that is adapted to rotate relative to a subject positioned therewithin. A control mechanism and associated control circuitry are typically provided for controlling operation of the CT system, e.g., rotation of the gantry, image capture and the like. Analog electrical signals are generated by the detector array and routed to an analog-to-digital converter which digitizes the analog signals. Thus, as the gantry is rotated, an angular range of projection views of the subject are obtained.

The disclosed CT system advantageously includes means for determining the energy dependency of the x-ray absorption process. By facilitating access to and use of such energy dependency information/data, the disclosed CT system facilitates the use of energy selective detection methods such as the Alvarez-Macovski approach, e.g., to improve contrast and/or image quality. The disclosed CT system addresses a fundamental prerequisite to effective use of energy selective detector measurements by quantifying the incident (filtered) x-ray tube spectrum to which the patient will be exposed in the CT system. Of note, the x-ray absorption processes of the human body spectrally modify the incident spectrum, thereby greatly complicating any effort to quantify the x-ray tube spectrum for a given patient.

The disclosed CT system permits quantification of x-ray tube spectra, thereby supporting Spectral CT imaging, by operating in conjunction with processing means that is adapted to run one or more programs to calculate x-ray tube spectra associated with a CT unit, or that is adapted to store and access x-ray tube spectra data from a database in communication with the processing means, or a combination thereof. The processing means may take the form of or include a central processing unit (CPU) of conventional design, and the CPU responsible for calculation of and/or access to the x-ray spectral data may be co-located with the CT unit (e.g., at the patient location) or may be in communication with the CT unit over a network, e.g., an intranet, extranet, local area network, wide area network or the like. Similarly, the data storage or computer memory in which the database(s) for housing x-ray spectral data may be co-located with the CT system, e.g., at a patient location, or may be remotely located and in communication with the processing means over a network, as described herein.

In an exemplary embodiment of the present disclosure, the processing means is adapted to support and run a spectral x-ray tube model calculation program. The model calculation program may take a variety of forms, as will be apparent to persons skilled in the art, and may include use of the output/results from Monte-Carlo simulations of the bremsstrahlung processes. In alternative embodiments of the present disclosure, the processing means is adapted to communicate with one or more spectra databases. The databases are populated with x-ray spectral data that may be derived in a variety of manners, e.g., data obtained experimentally, theoretically and/or by simulations. According to exemplary embodiments of the present disclosure, the x-ray spectral data within the spectra database(s) is periodically updated, e.g., at predetermined intervals. By updating the spectral data on a periodic basis, the disclosed CT system can effectively take account of changed conditions, e.g., aging effects of the x-ray tube.

In addition to supporting calculation of and/or access to x-ray spectral data, the disclosed processing means may also function, in whole or in part, as the controller for the CT unit. Thus, the processing means may perform such control functions as controlling the operation of the x-ray tube, the gantry and the data acquisition system (DAS).

The spectra determination systems and methods of the disclosed CT system advantageously mitigate the angular dependence of the tube spectra (e.g., the “heel” effect), particularly with respect to multi-slice CT scanners where the heel effect is most pronounced in the axial direction (parallel to the rotation axis of the gantry). Moreover, by determining and/or accessing x-ray spectral data for each CT system, the present disclosure provides an advantageous CT system architecture that supports energy selective preprocessing methods, e.g., the Alvarez-Macovski approach, and spectral CT imaging in general.

Additional features, functions and benefits of the disclosed CT system, CT system architecture and processing methods will be apparent from the detailed description which follows.

To assist those of ordinary skill in the art in making and using the disclosed CT systems and associated methods, reference is made to the accompanying figures, wherein:

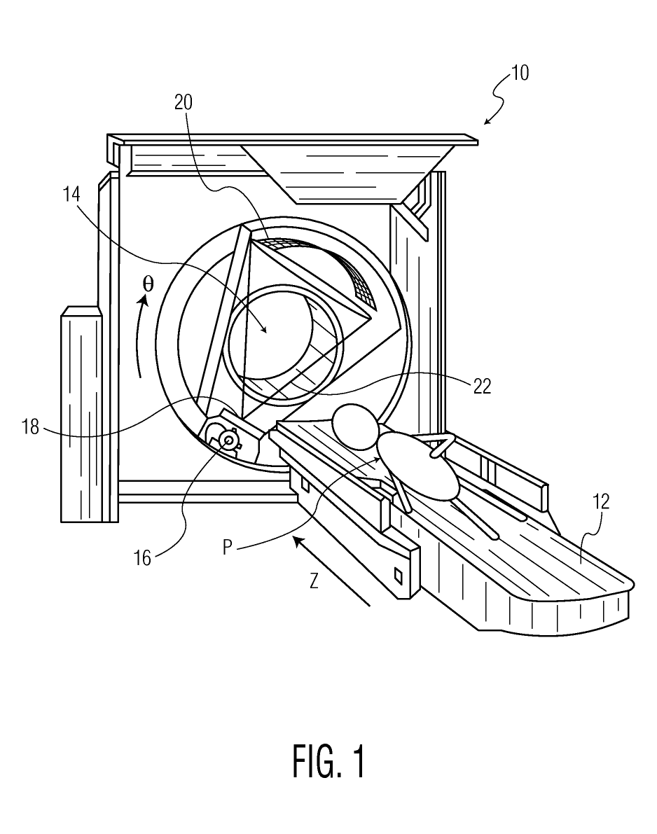

FIG. 1 is a schematic diagram of an exemplary computed tomography (CT) system for use according to the present disclosure;

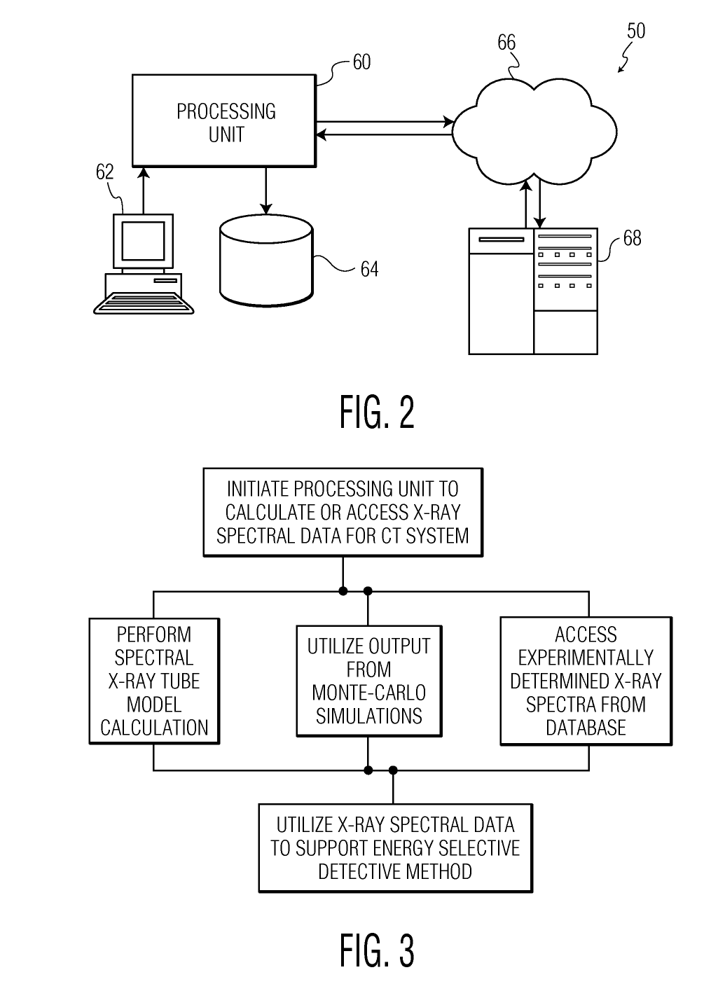

FIG. 2 is a schematic flowchart of data processing elements according to an exemplary embodiment of the present disclosure;

FIG. 3 is a flow chart of processing steps associated with the calculation and utilization of energy properties and/or x-ray tube spectra associated with a CT system according to the present disclosure.

The disclosed computed tomography (CT) systems utilize energy properties and/or x-ray tube spectra of CT units to enhance CT performance, e.g., in generating and/or interpreting CT data. The disclosed CT systems and methods are particularly adapted to use energy properties and/or x-ray tube spectra associated with CT systems to enhance contrast and/or image quality. According to exemplary embodiments of the present disclosure, the energy properties and/or x-ray tube spectra are used in support of energy selective preprocessing techniques, e.g., the Alvarez-Macovski approach, and the generation of CT images based on spectral information. The disclosed CT systems may be adapted to determine applicable energy properties and/or x-ray spectra in a variety of ways, e.g., through incorporation of a spectral x-ray tube model into the CT system, using the output of Monte-Carlo simulations, and/or processing measured experimental spectral tube data for the CT system.

With initial reference to FIG. 1, an exemplary CT system 10 is schematically depicted. CT system 10 includes an imaging subject support 12, such as a couch, which is linearly/axially movable along a Z-axis inside an examination region 14. An x-ray tube assembly 16 is mounted on a rotating gantry and is adapted to project x-rays through the examination region 14. A collimator 18 collimates the radiation in two dimensions. An x-ray detector array 20 is disposed on the rotating gantry across the examination region 14 from the x-ray tube assembly 16. In an alternative embodiment of the present disclosure, the x-ray detector array may take the form of non-rotating two-dimensional detector rings, e.g., detector rings that are mounted on a stationary gantry positioned around the rotating gantry. Detector array 20 generally includes a plurality of parallel detector rows of detector elements, such that projection data corresponding to a plurality of quasi-parallel slices can be acquired simultaneously during a scan.

The x-ray source generally projects a fan-shaped beam which is collimated to lie within an X-Y plane of a Cartesian coordinate system and generally referred to as an “imaging plane”. The x-ray beam passes through an object being imaged, such as a patient. The beam, after being attenuated by the object, impinges upon an array of radiation detectors. The intensity of the attenuated radiation beam received at the detector array is dependent upon the energy dependent attenuation of an x-ray beam by the object. Each detector element of the array produces a separate electrical signal that is a measurement of the beam intensity at the detector location. The intensity measurements from all the detectors are acquired separately to produce a transmission profile. A group of x-ray attenuation measurements, i.e., projection data, from the detector array at a particular gantry angle is referred to as a “view”.

With reference to FIG. 2, a schematic flowchart setting forth data processing elements is provided according to an exemplary embodiment of the present disclosure. The data processing elements are advantageously configured and adapted to process energy properties and/or x-ray tube spectra for a CT system, e.g., the exemplary CT system 10 of FIG. 1. Processing system 50 includes a processing unit 60 that functions as processing means according to the present disclosure. The processing unit 60 is typically a conventional computer system that has sufficient processing capabilities to perform the functions and support the operations described herein. For example, processing unit 60 may take the form of a personal computer or a workstation, although larger scale processing systems are also encompassed by the present disclosure, e.g., a minicomputer or distributed processing system. Processing unit 60 is generally adapted to receive input from an associated keyboard/monitor assembly 62. Thus, an operator is generally able to communicate instructions to processing unit from assembly 62, and receive/view results on the monitor associated with assembly 62. Although processing unit 60 and assembly 62 are schematically depicted as distinct components, the processing unit 60 may form an integrated part of assembly 62, as will be readily apparent to persons skilled in the art.

Processing unit 60 is further adapted to communicate with storage means or memory 64. As used herein, storage means 64 broadly encompasses the various types of computer storage available for database storage of data, e.g., internal and external disk storage, tape storage, etc. Although storage means 60 is schematically depicted as a distinct component relative to processing unit 60 and assembly 62, it is to be understood that storage means 60 may be an integrated aspect of either processing unit 60 or assembly 62, as will be readily apparent to persons skilled in the art.

With further reference to FIG. 2, processing unit 60 may be adapted to communicate with one or more remote computers/servers 68 across network 66. Network 66 may take the form of an intranet, extranet, local area network, wide area network or the like. According to exemplary embodiments of the present disclosure, network communications may include the transmission of information across the Internet to remote locations. Thus, according to network-based implementations of the present disclosure, the processing unit 60 may be adapted to communicate with computers/servers 68 that supply processing and/or memory capabilities thereto.

Turning to FIG. 3, the architecture and operation of the disclosed CT system are described in greater detail with reference to the flow chart provided therein. More particularly, the flow chart of FIG. 3 illustrates exemplary steps associated with the determination and utilization of x-ray spectral data in support of, for example, an energy selective preprocessing method, e.g., the Alvarez-Macovski approach. Thus, as shown in FIG. 3, a processing unit or processing means associated with a CT system is initiated to calculate or access x-ray spectral data. Processing unit initiation is generally undertaken through operator interaction with the system, e.g., through transmission of input/instructions to the processing unit.

Once initiated, the processing unit may obtain and/or access the x-ray spectral data for the CT system in a variety of ways. For example, as schematically depicted in FIG. 3, the processing unit may: (i) perform spectral x-ray tube model calculation(s), (ii) utilize output from Monte-Carlo simulations of the bremsstrahlung processes, and/or (iii) access experimentally determined x-ray spectra from one or more databases. With particular reference to the spectral x-ray tube model calculations, it is noted that the technical literature discloses exemplary x-ray models that may be employed according to the present disclosure, e.g., Tucker et al., “Semi-empirical model for generating tungsten target x-ray spectra,” Med. Phys. 18(2), 211, 1991 and Durand, “X-ray Generation Models,” PMS Report (1991), both of which are incorporated herein by reference.

In implementations of the present disclosure wherein x-ray spectral data has been determined experimentally, theoretically or by simulations, the disclosed CT system typically includes one or more databases that have been established/configured for electronic storage of such data. According to exemplary embodiments of the present disclosure, the x-ray spectral data within the spectra database(s) is periodically updated, e.g., at predetermined intervals. By updating the spectral data on a periodic basis, the disclosed CT system can effectively take account of changed conditions, e.g., aging effects of the x-ray tube.

Once obtained, the x-ray spectral data for the CT system is advantageously employed in support of further image-related processing, e.g., an energy selective preprocessing method. The disclosed determination and use of x-ray spectral data advantageously supports and/or facilitates spectral CT imaging. The use of x-ray spectral data—as determined and/or accessed herein—in connection with an energy selective detective method and generation of spectral CT images based thereon, is within the skill of persons of ordinary skill in the art.

The x-ray spectral data for a CT unit can vary based on a number of factors, including anode angle, anode material, tube voltage and the like. Thus, a number of different spectra exist. The disclosed system/system architecture and associated processing methodology advantageously determines/accesses such spectra for a given CT system and utilizes such x-ray spectral data in image generation. By facilitating access to and use of such energy dependency information/data, the disclosed CT system facilitates the use of energy selective preprocessing method, e.g., the Alvarez-Macovski approach, to improve contrast and/or image quality. Indeed, the disclosed CT system quantifies the x-ray tube spectra associated with the CT system, thereby supporting Spectral CT imaging.

In addition to supporting calculation of and/or access to x-ray spectral data, the disclosed processing means may also function, in whole or in part, as the controller for the CT unit. Thus, the processing means may perform such control functions as controlling the operation of the x-ray tube, the gantry and the data acquisition system (DAS).

The spectra determination systems and methods of the disclosed CT system advantageously mitigate the angular dependence of the tube spectra (e.g., the “heel” effect), particularly with respect to multi-slice CT scanners where the heel effect is most pronounced in the axial direction (parallel to the rotation axis of the gantry). Moreover, by determining and/or accessing x-ray spectral data for each CT system, the present disclosure provides an advantageous CT system architecture that supports energy selective detection methods and spectral CT imaging.

Of note, the disclosed CT system may also include a control mechanism and associated control circuitry for controlling operation of the CT system, e.g., rotation of the gantry, image capture and the like. Analog electrical signals are typically generated by the detector array and routed to an analog-to-digital converter which digitizes the analog signals. Thus, as the gantry is rotated, an angular range of projection views of the subject are obtained. The control mechanism associated with the disclosed CT system generally includes an x-ray controller that provides power and timing signals to the x-ray source and a gantry motor controller that controls the rotational speed and position of components on gantry. A data acquisition system (DAS) in the control mechanism samples analog data from the detector elements and converts the data to digital signals for subsequent processing. An image reconstructor receives sampled and digitized x-ray data from the DAS and performs high-speed image reconstruction. The reconstructed image is generally applied as an input to a computer, which stores the image in a storage device. The image reconstructor can take the form of specialized hardware and/or computer programs executing on the computer.

According to exemplary embodiments of the present disclosure, the control system and associated DAS are advantageously combined with the processing unit and associated data processing system described hereinabove. Thus, the computer associated with the data processing system may be adapted to receive commands and scanning parameters from an operator via a console that has a keyboard. An associated monitor allows the operator to observe the reconstructed image and other data from the computer. The operator-supplied commands and parameters are used by the computer to provide control signals and information to the DAS, x-ray controller, and/or gantry motor controller. In addition, the computer generally operates a table motor controller, which controls the imaging subject support to position the patient in the gantry.

Although the present disclosure has been described with reference to exemplary embodiments of the CT systems, system architectures and associated methods, the present disclosure is not limited to the exemplary embodiments disclosed herein. Rather, the disclosed systems and methods are susceptible to many modifications, variations and/or enhancements without departing from the spirit or scope of the present disclosure. The present disclosure expressly encompasses such modifications, variations and/or enhancements within the scope of hereof.

1. A computed tomography (CT) system, comprising:
a CT unit, and
processing means associated with the CT unit, the processing means being configured and adapted to determine and/or access x-ray spectral data associated with the CT unit.

2. A CT system according to claim 1, wherein the processing unit is configured and adapted to determine the x-ray spectral data using at least one spectral x-ray tube model.

3. A CT system according to claim 1, wherein the processing unit is configured and adapted to determine the x-ray spectral data using output from Monte-Carlo simulations.

4. A CT system according to claim 1, wherein the processing unit is configured and adapted to access x-ray spectral data that is stored in one or more databases.

5. A CT system according to claim 4, wherein the x-ray spectral data stored in the one or more databases was generated experimentally, theoretically and/or by simulations.

6. A CT system according to claim 1, wherein said processing means includes a central processing unit.

7. A CT system according to claim 6, wherein the central processing unit is adapted to communicate across a network with at least one remotely located computer/server.

8. A CT system according to claim 6, wherein said central processing unit is further adapted to provide one or more control functions to the CT unit.

9. A method for generating a computed tomography image, comprising:
providing a CT unit that includes an x-ray source and a detector array;
determining the x-ray spectral data associated with the CT unit; and
using the x-ray spectral data to enhance performance of the CT unit.

10. A method according to claim 9, wherein the x-ray spectral data is determined by using at least one spectral x-ray tube model.

11. A method according to claim 9, wherein the x-ray spectral data is determined by using output from Monte-Carlo simulations.

12. A method according to claim 9, wherein the x-ray spectral data is determined by accessing stored spectral data in one or more databases.

13. A method according to claim 12, wherein the stored spectral data was determined experimentally, theoretically and/or by simulations.

14. A method according to claim 12, further comprising updating the stored spectral data on a periodic basis.

15. A method according to claim 9, wherein the x-ray spectral data is used in connection with an energy selective detection method.

16. A method according to claim 9, wherein the x-ray spectral data is used in generating at least one spectral CT image.
